(19) United States
(12) Patent Application Publication
(10) Pub. No.: US 2015/0367094 A1
(43) Pub. Date: Dec. 24, 2015

(54) MEDICO-SURGICAL VIEWING DEVICES

(71) Applicant: SMITHS MEDICAL INTERNATIONAL LIMITED, Kent (GB)

(72) Inventors: Richard Hingley, Hythe (GB); Andrew Thomas Jeffrey, Hythe (GB); Stephen James Field, Canterbury (GB); Vincent Patrick McCory, Hythe (GB)

(73) Assignee: SMITHS MEDICAL INTERNATIONAL LIMITED, Kent (GB)

(21) Appl. No.: 14/379,004

(22) PCT Filed: Jan. 25, 2013
(86) PCT No.: PCT/GB2013/000028

§ 371 Date: Aug. 15, 2014

(30) Foreign Application Priority Data
Feb. 25, 2012 (GB) 1203388.2
Oct. 24, 2012 (GB) 1219086.4

(51) Int. Cl. A61M 16/04; B29C 65/64; A61B 1/05; A61B 1/00; A61B 1/267

(52) U.S. Cl. CPC A61M 16/0488; A61B 1/00124; A61B 1/267; A61B 1/00078; A61B 1/0011; A61B 1/053; B29C 65/64; A61M 2207/00; B29L 2031/7546

(57) ABSTRACT

A video viewing device 10, such as for use in inserting medical tubes 3, has an outer bendable sleeve 80 and an inner assembly comprising an electrical assembly 20 of a camera 11, cable 13 and connector 14 secured in a channel-shape support member 40. The inner assembly 20, 40 is threaded along the outer sleeve 80 so that the camera 11 is positioned at one end 12 and the connector 14 at its opposite end. The support member 40 is preferably made of two lengths 40A and 40B of different stiffness joined end to end with an angled butt join 42, the length towards the patient end 12 being more flexible so that the rear end of the viewing device 10 is stiffer than its patient end. An angled Coudé tip 121 is formed subsequently, the correct orientation of the camera 11 relative to the angle of the tip being achieved by viewing the image of a target 91, rotating the viewing device 10 until the output is at the desired orientation, then placing the device in an angled jig 93 and subjecting it to heat treatment to set the angle of the inclined tip.

This invention relates to medico-surgical viewing devices of the kind including an outer sleeve of a bendable material, an electrical assembly of an image sensor connected to one end of an electrical cable and an electrical connector connected to the opposite end of the cable.

The invention is more particularly concerned with viewing devices for use in placing apparatus within a patient such as inserting an endotracheal tube into the trachea. Traditional introducers or bougies take the form of a simple rod that can be bent to an approximate desired shape and can flex to accommodate the shape of the anatomy during insertion. The introducer may be made with an angled, Coudé tip to facilitate introduction. The introducer can be inserted more easily than the tube itself because it has a smaller diameter and can be bent and can flex to the ideal shape for insertion. The small diameter also gives the clinician a better view of the trachea around the outside of the introducer. These bougies may be used with or without the aid of a laryngoscope. When the bougie has been correctly inserted, a tube can be slid along its outside to the correct location, after which the bougie is pulled out of the tube, which is left in position. Bougies are available from Smiths Medical. GB2312378 describes an introducer or bougie moulded of an aliphatic polyurethane material and also describes an earlier bougie made from a braided polyester filament repeatedly coated in layers of resin. The characteristic flexural and recovery properties of these bougies are highly valued by clinicians.

More recently it has been proposed to use fibre optics or a video camera with an introducer to provide the clinician with a view of the trachea as the introducer is inserted. WO2007/089491 describes an arrangement with an introducer and a separate camera assembly clipped onto the outside of the introducer, which is removed before an endotracheal tube can be slid along the introducer. Alternative arrangements have a handle at the machine end of the introducer so that the apparatus has to be inserted with the tube already loaded on the introducer from its patient end. Such arrangements are more difficult to use because of the presence of the tube during insertion. WO2010/136748 describes an introducer arrangement with a camera at one end of an introducer and connected via a cable to a display screen at the opposite end. The introducer is disconnected from the display when correctly positioned so that an endotracheal tube can be slid along the introducer into position, following which the introducer is removed by pulling rearwardly from the endotracheal tube.

It is particularly important that these video introducers or bougies retain the flexural and recovery properties of conventional bougies, even after sterilisation and prolonged storage. If the diameter of the bore along the bougie has to be enlarged to accommodate the viewing means this can adversely affect the handling properties of the bougie. Another problem is that it can be difficult to connect the cable with an electrical connector at the rear end of the bougie. If the cable length is extended beyond the bougie to allow access for connection to a connector there can be surplus cable but no space within the bougie to receive this. Providing a video introducer or bougie at a reasonable cost with all the characteristics needed is not a trivial problem.

It is an object of the present invention to provide alternative an medico-surgical viewing device and method.

According to one aspect of the present invention there is provided a medico-surgical viewing device of the above-specified kind, characterised in that the assembly includes an inner support member with an outer diameter that enables it to be inserted within the outer sleeve, that the inner support member extends along the outside of the cable between the image sensor and the electrical connector to provide an inner assembly that can be inserted within the outer sleeve along its length so that the image sensor is located at one end of the outer sleeve and the electrical connector is located at the opposite end of the outer sleeve.

The inner support member is preferably in the form of a channel. The inner support member may be attached with the cable by means of an adhesive. The inner support member may have at least two regions along its length of different stiffness such that the viewing device has regions along its length of different stiffness. The inner support member is preferably arranged such that the viewing device is stiffer towards the rear, machine or connector end than the forward, patient or camera end. The inner support member is preferably formed from at least two lengths of material of different stiffness joined end to end. The join preferably extends longitudinally along a part of the length of the inner support member such that the proportion of materials of different stiffness varies along the join to give a gradual transition in stiffness along the length of the join. The join may be an angled butt join.

According to another aspect of the present invention there is provided a method of making a video introducer device including the steps of providing an inner assembly comprising an electrical assembly and an inner support member extending along the outside of the electrical assembly, the electrical assembly including an image sensor connected with an electrical cable at one end and having an electrical connector at the opposite end of the cable, threading the inner assembly along the length of an outer sleeve such that the image sensor is located at one end of the outer sleeve and the electrical connector is located at the opposite end of the outer sleeve, and securing the inner assembly in place within the outer sleeve.

The inner support member is preferably in the form of a channel with an opening along its length, the electrical assembly being placed in the channel through the opening. The inner support member may be secured with the electrical assembly after formation of the electrical assembly. The inner support member preferably has at least two regions along its length of different stiffness. The two regions may be provided by two lengths of material of different stiffness, the method including the step of joining the two lengths together end to end before threading along the outer sleeve. The two lengths may be joined together to form an angled butt join.

According to a further aspect of the present invention there is provided a method of forming an inclined tip at the patient end of a medico-surgical viewing device including the steps of providing a viewing device without an inclined tip, viewing on a screen an image from the device while angularly displacing the device about its axis until the desired orientation of the image on the screen is achieved, retaining the angular orientation of the device, bending the tip of the device while in the desired orientation and subjecting the bent tip of the device to treatment to set a desired incline in the bent tip.

Preferably the viewing device is placed on a flat surface and is angularly displaced by rolling the device on the surface. The viewing device is preferably placed in a jig to retain the bend of the tip in the desired orientation prior to treatment. The treatment may include heat treatment.

According to yet another aspect of the present invention there is provided a viewing device made by a method according to the above other or further aspect of the present invention.

An endotracheal tube placement or viewing device according to the present invention will now be described, by way of example, with reference to the accompanying drawings, in which:

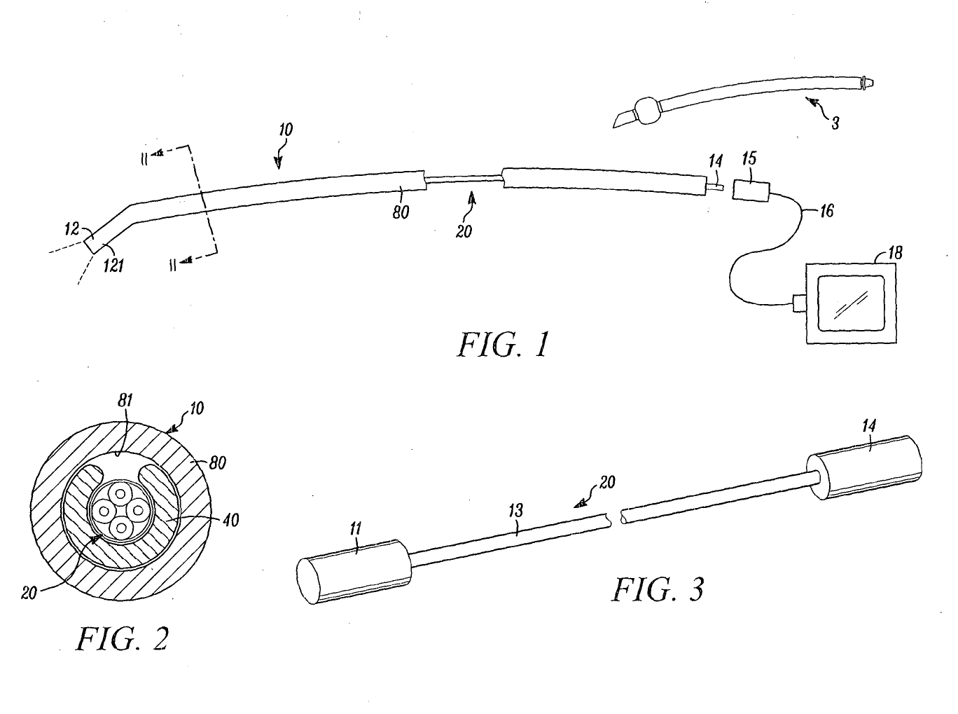

FIG. 1 shows an assembly of a viewing device in the form of a video introducer and an endotracheal tube;

FIG. 2 is an enlarged cross-section view of the device of FIG. 1 along the line II-II;

FIG. 3 is a perspective view of an inner electrical assembly with the inner support member omitted;

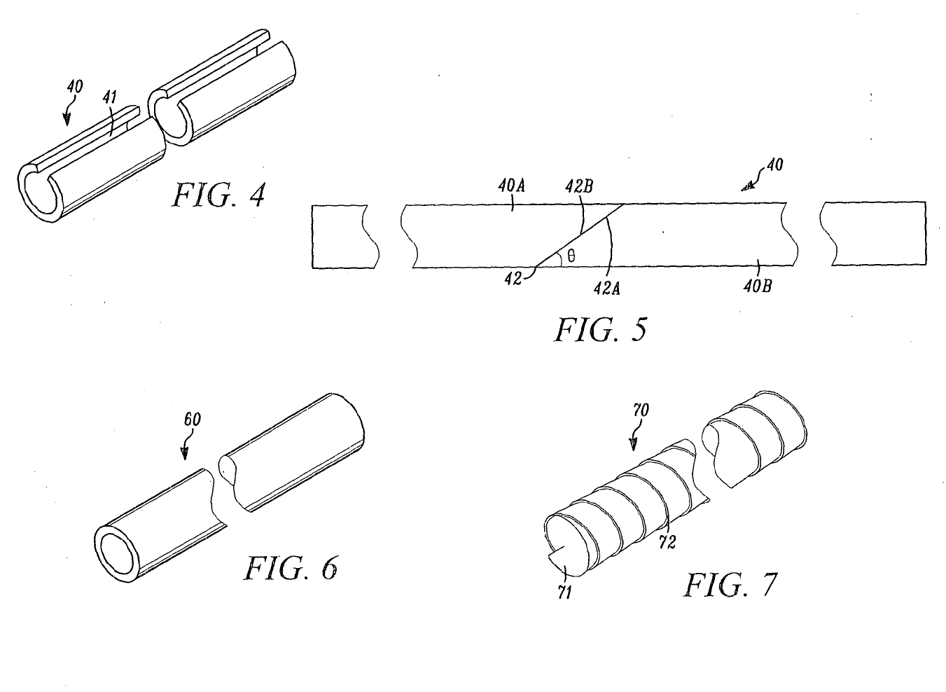

FIG. 4 is a perspective view of a first form of inner support member;

FIG. 5 is a side elevation view of the inner support member;

FIG. 6 is a perspective view of a second form of inner support member;

FIG. 7 is a perspective view of a third form of inner support member;

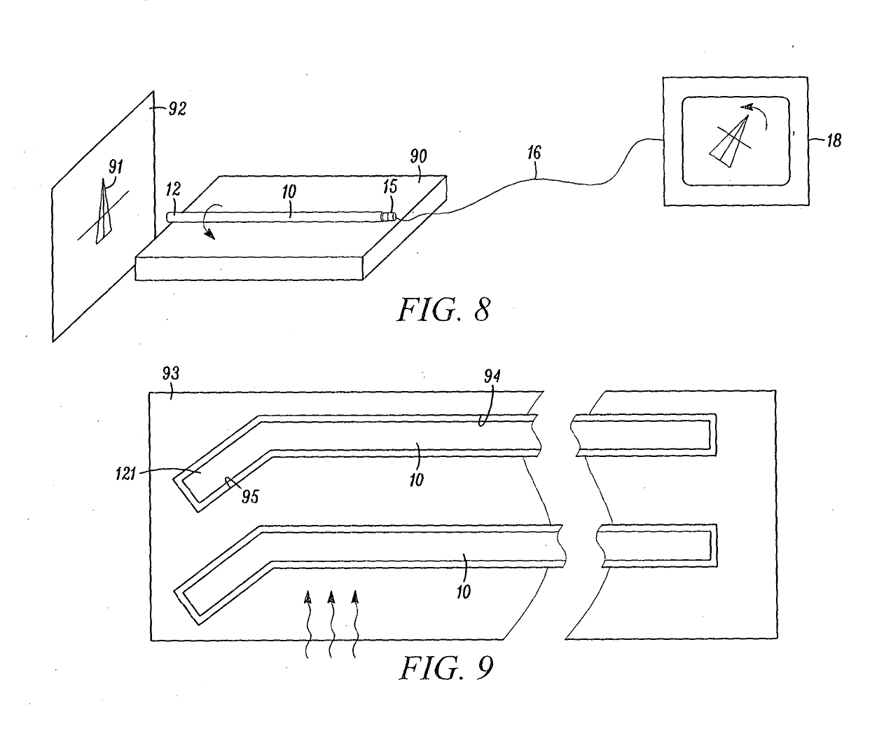

FIG. 8 is a perspective view illustrating a preliminary step in a method of forming an inclined Coudé tip on the viewing device; and

FIG. 9 is a plan view of a subsequent step in the method of forming the inclined tip.

With reference first to FIGS. 1 to 3, the viewing device is in the form of a video introducer 10 provided by a flexible, bendable rod or bougie with a small camera and illumination unit 11 mounted at its forward patient end 12. The camera and illumination unit 11 are connected with one end of an electrical cable 13 the opposite end of which is connected with a low profile electrical connector 14, which is small enough to allow an endotracheal tube 3 to be slid over the connector. The connector 14 mates removably with a connector 15 attached at one end of a second cable 16 extending to a video display monitor 18 on which is shown a display representation of the illuminated field of view of the camera unit 11.

The camera unit 11, cable 13 and connector 14 are pre-assembled as an electrical assembly sub-unit 20, as shown in FIG. 3. Because the connector 14 is connected with the cable 13 prior to insertion within the bougie 10 the assembly operation is straightforward and the cable can be provided of exactly the desired length. Testing of the electrical functions is also facilitated since this can be carried out before the sub-unit 20 is assembled.

The electrical assembly sub-unit 20 is then enclosed along the length of the cable 13 in an inner support member of the kind shown in FIG. 4, 6 or 7.

In its preferred form shown in FIGS. 4 and 5, the inner support member 40 is an open channel of C-shape in section extruded from a conventional plastics material with a slit, opening or gap 41 extending longitudinally parallel with the axis of the channel. The electrical assembly 20 can be inserted within the channel 40 laterally through the slit 41. The natural width of the slit 41 is slightly less than the diameter of the cable 13 so that the cable has to be compressed or the slit widened slightly during insertion of the cable. After inserting the cable 13 it expands to its natural diameter and the slit 41 closes to its natural width so that the cable is retained in the channel 40. The inside of the channel 40 is coated with an adhesive, such as an activated adhesive, so that the channel can be clamped about the cable 13 to allow the channel and cable to bond with one another and the adhesive to seep into and close the slit 41. Alternatively, the slit 41 could be closed by a fillet or a setting compound, or it could be left open. The length of the channel 40 is chosen so that it extends along the entire length of the cable 13 without covering the camera unit 11 or connector 14.

Preferably the channel 40 has two regions 40A and 40B (FIG. 5) along its length of different stiffness. More particularly, the region 40A towards the forward, patient or camera end 12 is softer or more flexible than the region 40B towards the rear end. The reason for this variation in stiffness is to provide a stiffer rear end in order to help support the weight of the cable 16 attached to the rear of the introducer 10 but without adversely affecting the flexure and handling properties of the forward patient end 12 manipulated by the clinician. The ratio of the length of the flexible forward portion 40A to that of the stiffer rear portion 40B is preferably about 2:5, with the forward portion being about 200 mm long. In the present example, the two regions 40A and 40B are formed by separate lengths of plastics materials having different stiffness, preferably provided by different grades of the same plastics material so as to facilitate joining the two lengths together. FIG. 5 shows the two lengths 40A and 40B joined end to end by an angled butt join 42 with an angle θ of about 60°, the two inclined ends 42A and 42B being bonded by a suitable solvent, adhesive or by a thermal bond. The angled or inclined join 41 has the advantage of providing a greater surface area for the bond to increase its integrity but, more importantly, it results in a gradual variation along the length of the join of the proportion of soft material (from region 40A) to harder material (from region 40B), the amount of harder material increasing gradually rearwardly along the join. This makes for a less abrupt perceived change of stiffness of the introducer 10 in the region of the join 42. Various alternative configurations of join are possible. It is not essential that the inner support member 40 varies in stiffness along its length although this is desirable. The support member could be provided with more than two regions of different stiffness, or it could have a progressively varying stiffness along its length. In particular, the region around the bend of the Coudé tip 121 at the patient end 12 could be of a more flexible material than the remainder of the forward patient end region 40A. There are many alternative ways of varying the stiffness of the support member along its length. For example, the support member could be extruded with two layers of materials of different stiffness that vary in thickness along the length of the support member, or the support member could be made from a blend of two materials of different stiffness, the proportion of the blend varying along its length. Alternatively, the support member could be of the same material along its length and could be perforated with holes the number or size of which varies along its length.

The inner support member need not be of channel shape but could be in the form of a heat-shrink sleeve 60, such as shown in FIG. 6, of the kind that is manufactured with a first diameter and can be reduced to a second, smaller diameter by the application of heat, such as from a hot-air blower. To form an inner assembly using the sleeve 60, this is provided with a natural internal diameter that is large enough to be slid over either the camera unit 11 or the connector 14 so that the sleeve can be slid onto the electrical assembly from one end. Heat is then applied to shrink the sleeve 60 securely along the length of the cable 13, leaving the camera unit 11 and connector 14 exposed at opposite ends. Such a sleeve 60 could be arranged to have regions of different stiffness along its length as in the channel 40 of FIGS. 4 and 5.

FIG. 7 shows another alternative inner support member 70 in the form of a helical strip 71 wound to form a tube with a helical slit 72, which can be opened to allow the cable 13 of the electrical assembly 20 to be inserted by stretching or unravelling the strip. The natural resilience of the strip 71 allows it to return to a tubular configuration when released. The inside of the tube 70 is coated with an adhesive so that it bonds securely with the outside of the cable 13. Again, the strip 71 could have regions of different stiffness along its length.

A conventional endotracheal bougie has an external diameter of about 5 mm and a bore with a diameter of about 2 mm. In the arrangement of the present invention the bougie 10 has an outer sleeve 80 of a bendable plastics material with the same external diameter of 5 mm but the bore 81 is enlarged to about 4 mm in order to receive the inner assembly of the electrical assembly 20 and the inner support member 40, 60 or 70. The external diameter of the inner support member 40, 60 or 70 is chosen to be a close sliding fit within the bore 81 of the outer sleeve 80 so that the inner assembly can be slid into position along the length of the outer sleeve with the camera 11 positioned at one end and the connector 14 positioned at the opposite end. An adhesive is coated on the outside of the inner support member 40, 60 or 70 before insertion, both to lubricate the passage along the bore 81 and to secure the inner assembly in the outer sleeve after curing of the adhesive. The electrical assembly 20 with the inner support member 40, 60 or 70 is sufficiently stiff to allow it to be pushed along the bore 81 of the outer tube 80.

Although the wall thickness of the outer sleeve 80 is reduced compared with the wall thickness of conventional bougies, the combined effect of the inner support member and outer sleeve can be arranged to give the same handling properties as conventional endotracheal bougies.

The introducer 10 is formed with an inclined Coudé tip 121 after the inner assembly has been inserted and bonded in the outer sleeve 80, which is naturally straight. The Coudé tip 121 must be inclined appropriately relative to the orientation of the camera 11 so that the image formed on the monitor 18 is correctly oriented. The usual way in which this is done is to provide the camera unit with some form of orientation marking and to use this as a guide when bending the tip of the introducer. However, the reduction in the size of video camera chips in recent years means that such orientation markings are now very small, making them difficult to view and to orientate accurately. Accordingly, a preferred alternative method is illustrated in FIGS. 8 and 9. In this method the introducer 10, in a straight form, is connected to the video display monitor 18 and is laid on a flat surface 90, such as a table top. The patient end 12 of the introducer 10 is pointed towards an alignment target 91 provided by printed markings on a screen 92. The image of the alignment target 91 on the video monitor 18 is viewed and the introducer 10 is rolled by hand slowly a small distance across the surface 90 to alter the angular orientation of the introducer and hence its camera unit 11 until the target as viewed on the monitor 18 is in the desired orientation. The introducer 10 is then lifted from the table top 90, while maintaining its orientation, and is transferred to a tip-forming jig 93 as shown in FIG. 9. The jig 93 has a channel 94 that is straight along a major part of its length but has a short angled section 95 at one end arranged to retain the patient end tip 12 of the introducer in the desired angle of the Coudé tip 121. The jig 93 has channels 94 for retaining several introducers although FIG. 9 only shows two such channels for simplicity. The jig 93 and its loaded introducers 10 is then subjected to a suitable treatment cycle so that the bent tips 12 of the introducers are given a permanent set with the desired angled Coudé tip 121. Typically this treatment would involve some kind of heat treatment, such as a cycle of warming to soften and relax the plastics material of the inner support member 40 and the outer sleeve 80, followed by a cycle of cooling so that the tip 12 of the introducer 10 takes on permanently the angle defined by the angled section 95 in the jig 93.

Instead of forming the Coudé tip after inserting the channel 40 or other support member in the outer sleeve 80 it would be possible to form the Coudé tip in the channel alone before inserting it in the sleeve. The channel 40 would straighten during insertion into the sleeve 80. When the channel 40 or other support member has been fully inserted, the relatively thin sleeve 80 would relax and take the bend of the channel so that the completed assembly takes the desired bend.

The finished introducers are then packaged and sterilised. It may be necessary for the introducers to be packaged with some form of retainer to hold the tip bent in the Coudé tip shape since, after prolonged storage the bent tip could relax and straighten especially if stored in warm conditions.

The arrangement of the present invention greatly facilitates cost-effective manufacture of a reliable video introducer.

1-19. (canceled)

20. A medico-surgical viewing device including an outer sleeve of a bendable material, an electrical assembly of an image sensor connected to one end of an electrical cable and an electrical connector connected to the opposite end of the cable, characterized in that the assembly includes an inner support member with an outer diameter that enables it to be inserted within the outer sleeve, that the inner support member extends along the outside of the cable between the image sensor and the electrical connector to provide an inner assembly that can be inserted within the outer sleeve along its length so that the image sensor is located at one end of the outer sleeve and the electrical connector is located at the opposite end of the outer sleeve.

21. A device according to claim 20, characterized in that the inner support member is in the form of a channel.

22. A device according to claim 20, characterized in that the inner support member is attached with the cable by means of an adhesive.

23. A device according to claim 20, characterized in that the inner support member has at least two regions along its length of different stiffness such that the viewing device has regions along its length of the different stiffness.

24. A device according to claim 23, characterized in that the inner support member is arranged such that the viewing device is stiffer towards the rear, machine or connector end than the forward, patient or camera end.

25. A device according to claim 23, characterized in that the inner support member is formed from at least two lengths of material of different stiffness joined end to end.

26. A device according to claim 25, characterized in that the join extends longitudinally along a part of the length of the inner support member such that the proportions of material of different stiffness varies along the join to give a gradual transmission in stiffness along the length of the join.

27. A device according to claim 26, characterized in that the join is an angled butt join.

28. A method of making a video introducer device including the steps of providing an inner assembly comprising an electrical assembly and an inner support member extending along the outside of the electrical assembly, the electrical assembly including an image sensor connected with an electrical cable at one end and having an electrical connector at the opposite end of the cable, threading the inner assembly along the length of an outer sleeve such that the image sensor is located at one end of the outer sleeve and the electrical connector is located at the opposite end of the outer sleeve, and securing the inner assembly in place within the outer sleeve.

29. A method according to claim 28, characterized in that the inner support member is in the form of a channel with an opening along its length, and that the electrical assembly is placed in the channel through the opening.

30. A method according to claim 28, characterized in that the inner support member is secured with the electrical assembly after formation of the electrical assembly.

31. A method according to claim 28, characterized in that the inner support member has at least two regions along its length of different stiffness.

32. A method according to claim 31, characterized in that the two regions are provided by two lengths of materials of different stiffness, and that the method includes the step of joining the two lengths together end to end before threading along the outer sleeve.

33. A method according to claim 32, characterized in that the two lengths are joined together to form an angled butt join.

34. A method of forming an inclined tip at the patient end of a medico-surgical viewing device including the steps of providing a viewing device without an inclined tip, viewing on a screen an image from the device while angularly displacing the device about its axis until the desired orientation of the image on the screen is achieved, retaining the angular orientation of the device, bending the tip of the device while in the desired orientation and subjecting the bent tip of the device to treatment to set a desired incline in the bent tip.

35. A method according to claim 34, characterized in that the viewing device is placed on a flat surface and is angularly displaced by rolling the device on the surface.

36. A method according to claim 34, characterized in that the viewing device is placed in a jig to retain the bend of the tip in the desired orientation prior to treatment.

37. A method according to claim 34, characterized in that the treatment includes heat treatment.

38. A viewing device made by a method according to claim 28.

39. A viewing device made by a method according to claim 34.
